(19) United States
(12) Patent Application Publication
(10) Pub. No.: US 2004/0157811 A1
(43) Pub. Date: Aug. 12, 2004

(54) Inhibition of the growth factor dependency of tumor cells

(76) Inventors: Rosemarie Lichtner, Berlin (DE); Ulrike Fuhrmann, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/399,318

(22) PCT Filed: Oct. 17, 2001
(86) PCT No.: PCT/EP01/12004

(30) Foreign Application Priority Data
Oct. 18, 2000 (DE) 100 51 609.2

(51) Int. Cl. A61K 31/57

(52) U.S. Cl. 514/177

(57) ABSTRACT

The invention relates to the use of progesterone receptor inhibitors for inhibition of growth-factor-dependency of tumor cells.

The invention relates to the use of progesterone receptor inhibitors for inhibiting the growth factor dependency of tumor cells.

Estradiol and progesterone are involved in the development of breast cancer. At the time of diagnosis, however, only about ⅓ of the tumors show a steroid hormone dependency. It is assumed that in the majority of steroid hormone-resistant tumors, the proliferation control for local-acting autocrine or paracrine peptidic growth factors is taken over. In this case, invasive tumors with extremely poor prognosis that are growth-factor-receptor-positive and steroid hormone-resistant result (Elledge et al., Semin. Onkol. 19 (1992), 244-253).

Growth factors regulate the cell growth by activation of intracellular signal transduction pathways after binding to highly affine tyrosine kinase receptors on the cell surface. More recent findings suggest that breast carcinoma cells can be sensitized by progestins for the mitogenic action of EGF (Groshong et al., Mol. Endocrinol. 11 (1997), 1593-1607). Thus, for example, it was possible for progesterone in the human breast carcinoma cell line T47D to induce the onset of cells in the S-phase accompanied by a transient increase of the activity of cyclin 1D and the cyclin-dependent kinase 4. The growth stimulation is limited to a single cycle, however, and is followed by a growth arrest at the G1/S-transition of the second cycle (Groshong et al. (1997), supra: Musgrove et al., Mol. Cell. Biol. 13 (1993), 3577-3587). In its condition that is stopped by progesterone, the cells are sensitive to the proliferative action of EGF. In addition, it was shown that progesterone enhances the action of EGF on T47D cells by ramping up EGFR, Erb2 and Erb3 and increases the tyrosine phosphorylation of signal molecules (Lange et al., J. Biol. Chem. 273 (1998), 31308-31316; Richer et al., J. Biol. Chem. 273 (1998), 31317-31326). In contrast, it has not yet been possible to show an inhibition of the action of EGF on tumor cells by influencing the progesterone receptor.

Within the scope of tests leading to this invention, it has now been found, surprisingly enough, that inhibitors of the progesterone receptor, e.g., 17α-fluoroalkyl steroids, can at least partially inhibit the binding of growth factors, such as EGF, to tumor cells, especially to tumor cells that have a high and/or constitutive expression of the progesterone receptor.

A subject of this invention is thus the use of an inhibitor of the progesterone receptor for the production of an agent for inhibiting the binding of growth factors to tumor cells and especially for inhibiting a proliferation of tumor cells or tumors that are produced by growth factors. An inhibitor of the progesterone receptor in terms of this invention is preferably a substance that competitively inhibits the binding of progesterone to its receptor. In this case, the inhibitor of the progesterone receptor is preferably selected from 17α-fluoroalkyl steroids, as they are disclosed in, e.g., WO98/34947. These 17α-fluoroalkyl steroids exhibit general formula I:

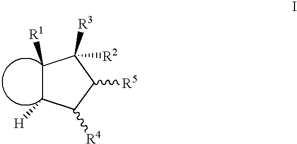

in which

R¹ stands for a methyl or ethyl group,

R² stands for a radical of formula CₙFₘHₒ, whereby n=2, 3, 4, 5 or 6, m>1 and m+o=2n+1,

R³ stands for a free, etherified, or esterified hydroxy group,

R⁴ and R⁵ each stand for a hydrogen atom, together for an additional bond or a methylene group,

St stands for a steroidal ABC-ring system of partial formula A, B or C

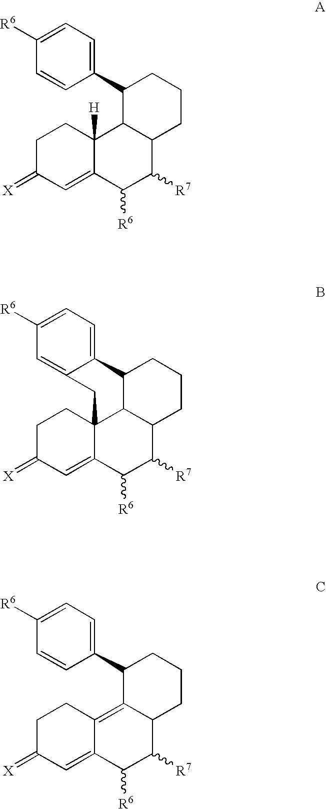

in which

R⁶ means a hydrogen atom, a straight-chain C₁-C₄ alkyl group or a branched C₃-C₄ alkyl group or a halogen atom,

R⁷ means a hydrogen atom, a straight-chain C₁-C₄ alkyl group or a branched C₃-C₄ alkyl group, or, if St stands for a steroidal ABC-ring system A or B, in addition R⁶ and R⁷ together mean an additional bond,

X means an oxygen atom, a hydroxymino grouping ═N—OH or two hydrogen atoms,

R⁸ means a radical Y or an aryl radical that is optionally substituted with a group Y in several places,

whereby Y is a hydrogen atom, a halogen atom, an —OH, —NO₂, —N₃, —CN, —NR^{9a}R^{9b}, —NHSO₂R⁹, —CO₂R⁹, C₁-C₁₀ alkoxy, C₁-C₁₀ alkanoyloxy, benzoyloxy-C₁-C₁₀ alkanoyl, C₁-C₁₀ hydroxyalkyl or benzoyl group,

and R^{9a} and R^{9b} are the same or different and like R⁹ represent a hydrogen atom or a C₁-C₁₀ alkyl group,

and for radicals —NR^{9a}R^{9b}, also their physiologically compatible salts with acids and for radicals —CO₂R⁹ with R⁹ in the meaning of hydrogen also their physiologically compatible salts with bases.

An especially preferred example of such inhibitors of the progesterone receptor is the compound 11β-(4-acetylphenyl)-17β-hydroxy-17α-(1,1,2,2,2-pentafluoroethyl)-estra-4,9-dien-3-one (compound A below). Moreover, other antiprogestins, for example onapristone (11β-[p-(dimethylamino)phenyl]-17α-hydroxy-17-(3-hydroxypropyl)-13α-estra-4,9-dien-3-one) are also suitable, however.

The action of the progesterone receptor inhibitors is found especially in the case of tumor cells that have a high and/or constitutive progesterone receptor expression, for example the progesterone receptor-positive breast carcinoma cell line T47D (Sartorius et al., Cancer Res. 54 (1994), 3668-3877).

The progesterone receptor inhibitors inhibit the progesterone-induced enhancement of the expression of growth factors, especially those factors that bind to growth factors of the EGF receptor family, such as, for example, the EGF receptor. The inhibitors especially preferably inhibit the binding of EGF to human breast carcinoma cells.

According to this invention, the progesterone receptor inhibitors can therefore be used for tumor therapy in mammals and preferably in humans, specifically especially to block the progression of a tumor, especially a breast carcinoma of steroid-dependent growth to growth-factor-dependent growth. In this way, an effective treatment of the tumor can take place in the stage of the steroid-dependent growth, e.g. by antiestrogens, without the tumor being able to progress in the stage of the growth-factor-dependent growth, associated with a considerable worsening of the prognosis for the patient. The administration of the progesterone receptor inhibitors can also produce a slowing of tumor growth in the stage of the growth-factor-dependent growth.

For the purpose of this invention, non-steroidal antiestrogens, such as, e.g., tamoxifen and nafoxidine, and raloxifene and EM800, can be used. The two last-mentioned antiestrogens are representatives of the thus mentioned SERMs (Selective Estrogen Receptor Modulators); also, other compounds with the profile of action of the SERMS can be used according to the invention, e.g., the compounds that are mentioned in PCT/EP99/05093 and of the latter in turn especially the compound 5-(4-{5-[(RS)-4,4,5,5,5-pentafluoropentyl)sulfinyl]-pentyloxy}phenyl)-6-phenyl-8,9-dihydro-7H-benzocyclohepten-2-ol.

Examples of steroidal antiestrogens comprise those that are disclosed in EP 0 348 341 A, especially Faslodex, and those that are disclosed in WO98/07740, especially 11β-fluoro-7α-{5-[N-methyl-N-3-(4,4,5,5,5-pentafluoropentylthio-propylamino]-pentyl}-estra-1,3,5(10)triene-3,17β-diol, or those that are described in WO99/33855, especially 11β-fluoro-7α-(5-[methyl-(7,7,8,8,9,9,10,10,10-nonafluoro-decyl)-amino]-pentyl)-estra-1,3,5(10)triene-3,17β-diol or pharmaceutically compatible derivatives or analogs thereof. Aromatase inhibitors with an antiestrogenic effect, such as, for example, those that are known from pages 7-8 of EP 0 495 825 B1, can likewise be used as antiestrogens.

The administration of the progesterone receptor inhibitors can be carried out according to commonly used methods, for example locally, topically, subcutaneously, enterally or parenterally. For enteral administration, especially tablets, coated tablets, capsules, pills, suspensions or solutions are suitable, which can be produced in the usual way with the additives and vehicles that are known in gallenicals. For local or topical use, for example, vaginal suppositories or transdermal systems such as skin patches are suitable. The subcutaneous administration can be carried out by injection with an oily solution.

A dosage unit can contain, for example, 0.1 to 100 mg of active compound(s) (=inhibitor(s) of the progesterone receptor). For administration in humans, the daily dose of the active compound(s) is approximately 0.1 to 400 mg, preferably approximately 10-100 mg and especially approximately 50 mg.

In addition, the invention is to be explained by the following examples and figures. Here:

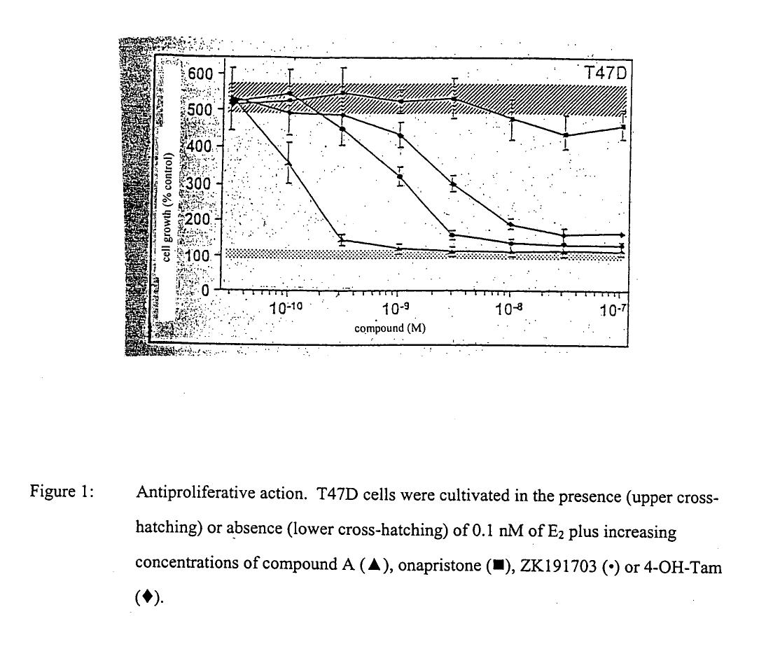

FIG. 1 shows the antiproliferative action of test substances on the breast carcinoma cell line T47D.

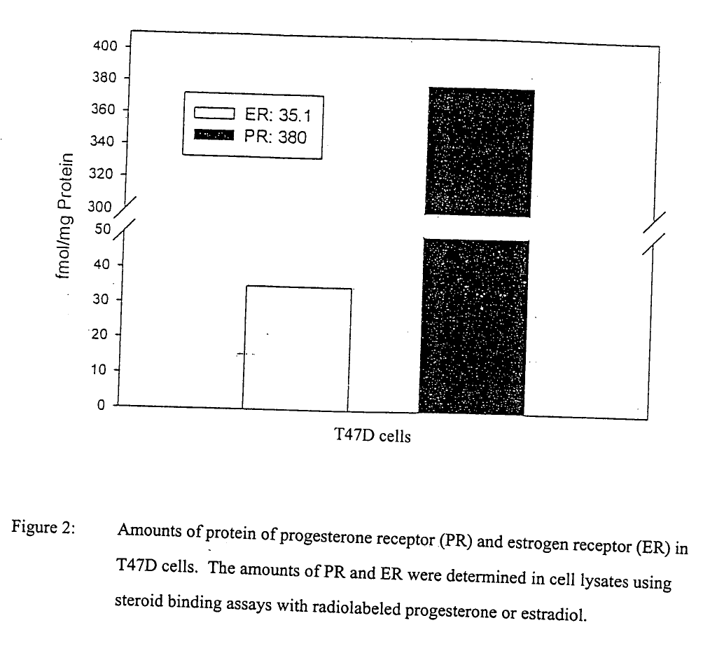

FIG. 2 shows the amounts of protein of progesterone receptor (PR) and estrogen receptor (ER) in breast carcinoma cell line T47D.

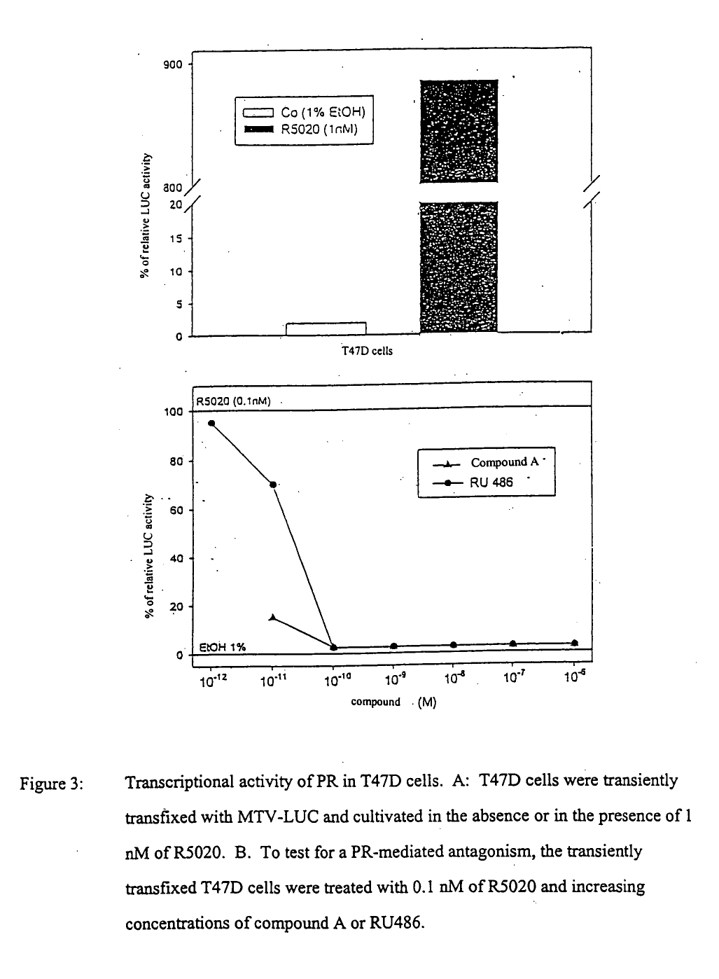

FIG. 3 shows the transcriptional activity of the progesterone receptor in T47D cells.

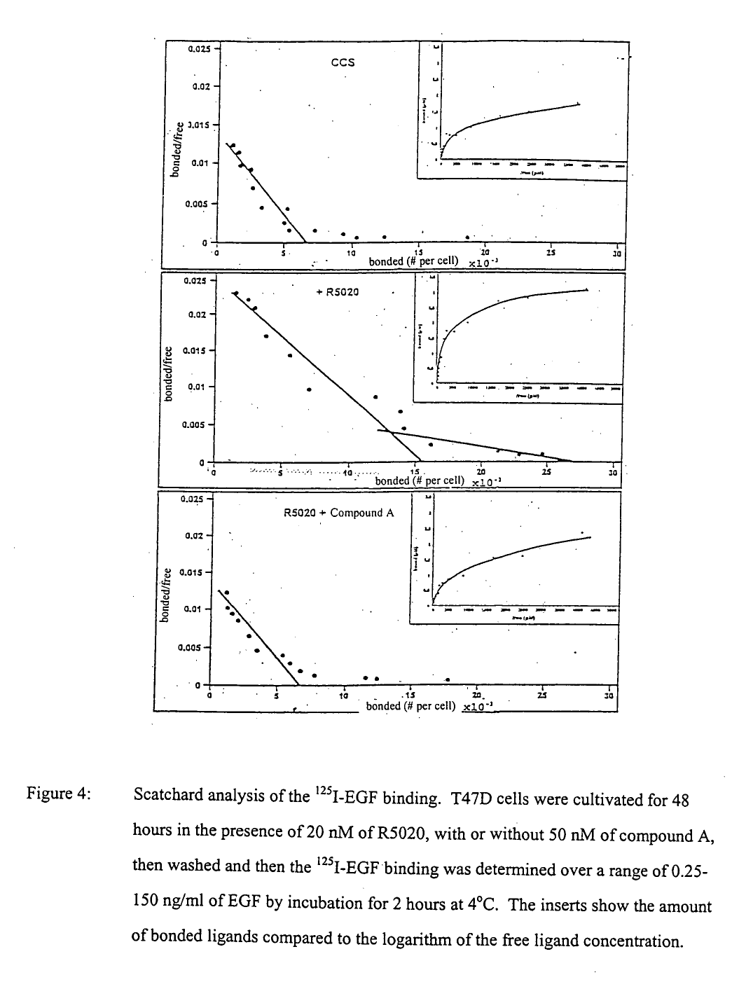

FIG. 4 shows a Scatchard analysis of the binding of EGF to T47D cells as a function of the presence of test substances.

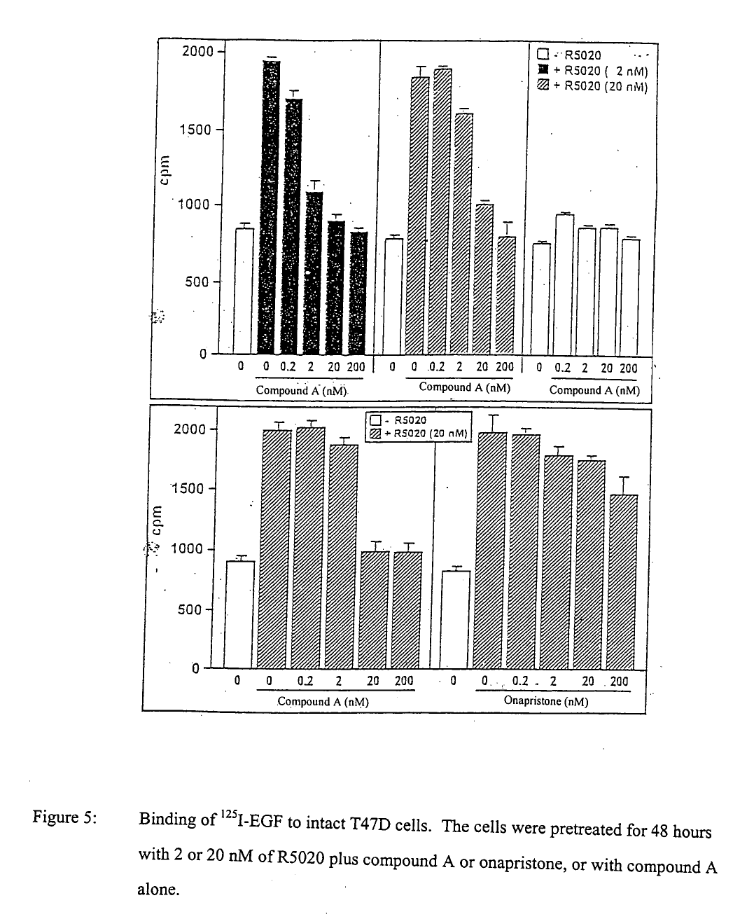

FIG. 5 shows the dependence of the binding of EGF to T47D cells on the presence of test substances.

### EXAMPLE

1. Materials and Methods

Materials:

¹²⁵I-EGF (100 mCi/mmol) was obtained by Amersham Buchler. Compound A, hydrotamoxifen (4-OH-Tam), ZM182780 and estradiol were synthesized in the Institut für Arzneimittelchemie [Institute for Pharmaceutical Agent Chemistry] of the Schering AG according to known methods.

Cell Lines:

The human estrogen receptor (ER)- and progesterone receptor (PR)-positive breast carcinoma cell line T47D (Freake et al., BBRC 101 (1981), 1131-1138) was used.

Growth Studies:

The tumor cells were cultivated at 5000 cells/well in 96-well plates for 6 days in RPMI medium plus 10% bovine serum, 200 nM of insulin and 0.1 nM of estradiol in the presence of the compounds that are indicated in each case, and the growth was determined by staining with crystal violet.

Amount of PR and ER Protein:

The amounts of PR and ER in cell lysates are determined with use of steroid binding assays with radiolabeled progesterone or estradiol according to methods described in Fuhrmann et al. (Contraception 54 (1996), 243-251).

Binding of ¹²⁵I-EGF to Tumor Cells:

R5020-Pretreated T47D cells were incubated for 2 hours with ¹²⁵I-EGF at 4° C. The unspecific binding was always less than 10% of the total binding.

Transactivation Assay:

T47D cells were transiently transfixed with MTV-LUC (Cato et al., EMBO J., 9: 2237-40) and cultivated in the absence or the presence of 1 nM of R5020. In the test on a PR-mediated antagonism, the transiently transfixed T47D cells were treated with R5020 and in addition with increasing concentrations of compound A or RU486. After 24 hours, a luciferase test was performed.

2. Results FIG. 1 shows the antiproliferative action of various test substances. T47D cells were cultivated in the presence (upper cross-hatching) or absence (lower cross-hatching) of 0.1 nM of E₂ plus increasing concentrations of compound A (▴), onapristone (▪), ZK191703 (●) or 4-OH-Tam (♦). In the case of T47D cells, compound A also shows a significant antiproliferative action at extremely small concentrations.

FIG. 2 shows the amounts of PR- and ER protein in T47D cells.

FIG. 3 shows the transcriptional activity of PR in T47D cells, whereby the respective cells were transiently transfixed with MTV-LUC and cultivated (a) in the absence of (Co) or the presence of 1 nM of R5020. In the test for a PR-mediated antagonism, the transiently transfixed T47D cells were treated with 0.1 nM of R5020 and increasing concentrations of compound A or RU468 (b).

In FIG. 4, a Scatchard analysis of the ¹²⁵I-EGF binding to T47D cells is shown. The cells were cultivated for 48 hours in the presence of 20 nM of R5020 with or without 20 nM of compound A and then washed. Then, the EGF-binding over a concentration range of 0.25 to 150 ng/ml of EGF was determined by incubation for 2 hours at 4° C. The insertions show the amount of bonded ligands relative to the logarithm of the free ligand concentration. It is clear that it was possible to block the increase of the EGF binding that is caused by R5020 (middle figure) relative to monitoring (upper figure) when compound A (lower figure) is added.

In FIG. 5, the binding of ¹²⁵I-EGF to intact T47D cells is shown. For this purpose, the cells were treated for 48 hours with 2 or 20 nM of R5020 plus compound A or onapristone or compound A alone. It can also be seen here that compound A blocks the increase of the EGF-binding to T47D cells caused by R5020. A similar—although considerably weaker effect—is also found for onapristone.

3. Discussion

The above results show that the estradiol-stimulated growth of T47D cells with high and constitutive PR contact was effectively blocked by compound A.

By transactivation assays, it was possible to show that the PR was transcriptionally active in the T47D cells and could be blocked by compound i.

A stimulation of the T47D cells with R5020 resulted in a 2× to 3×-increased EGF-receptor expression, which was blocked by compound A. At the same time, the binding of EGF to the cells was increased 2- to 3-fold and could be prevented by compound A and less efficiently by onapristone. The increased EGF-binding to R5020-treated cells could be produced by an enhanced EGF-receptor expression or increased heterodimer formation between the EGF receptor and erbB2.

These results show the interactions between PR- and growth factor-signal systems in human breast carcinoma cells. By use of antiprogestins, the progression of tumor cells from steroid-dependent growth is inhibited or prevented for growth-factor-dependent growth.

1. Use of an inhibitor of the progesterone receptor for the production of an agent for the inhibition of the binding of growth factors to tumor cells and/or to a tumor.

2. Use according to claim 1, characterized in that a proliferation of the tumor cells and/or the tumor produced by growth factors is inhibited.

3. Use according to claim 1 or 2, wherein the inhibitor of the progesterone receptor is selected from 17α-fluoroalkyl steroids of general formula I 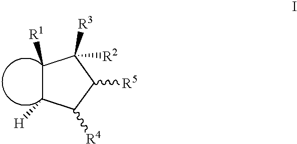
in which
R¹ stands for a methyl or ethyl group,
R² stands for a radical of formula CₙFₘHₒ, whereby n is 2, 3, 4, 5 or 6, m>1 and m+o=2n+1,
R³ stands for a free, etherified, or esterified hydroxy group,
R⁴ and R⁵ each stand for a hydrogen atom, together for an additional bond or a methylene group,
St stands for a steroidal ABC-ring system of partial formula A, B or C 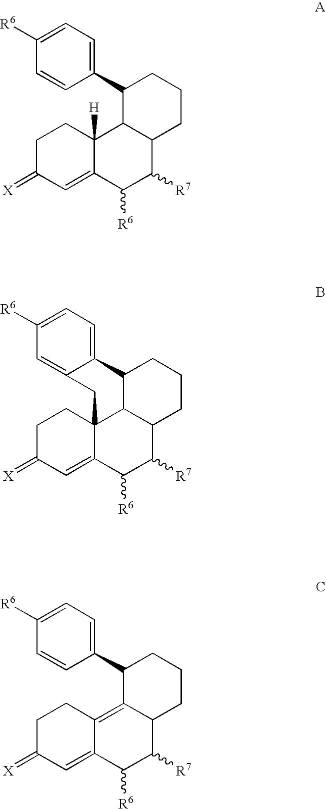
in which
R⁶ means a hydrogen atom, a straight-chain C₁-C₄ alkyl group or a branched C₃-C₄ alkyl group or a halogen atom,
R⁷ means a hydrogen atom, a straight-chain C₁-C₄ alkyl group or a branched C₃-C₄ alkyl group, or, if St stands for a steroidal ABC-ring system A or B, in addition R⁶ and R⁷ together mean an additional bond,
X means an oxygen atom, a hydroxymino grouping ═N—OH or two hydrogen atoms,
R⁸ means a radical Y or an aryl radical that is optionally substituted with a group Y in several places, whereby Y is a hydrogen atom, a halogen atom, an —OH, —NO₂, —N₃, —CN, —NR^{9a}R^{9b}, —NHSO₂R⁹, —CO₂R⁹, C₁-C₁₀ alkoxy, C₁-C₁₀ alkanoyloxy, benzoyloxy-C₁-C₁₀ alkanoyl, C₁-C₁₀ hydroxyalkyl or benzoyl group,
and R^{9a} and R^{9b} are the same or different and like R⁹ represent a hydrogen atom or a C₁-C₁₀ alkyl group,
and for radicals —NR^{9a}R^{9b}, also their physiologically compatible salts with acids and for radicals —CO₂R⁹ with R⁹ in the meaning of hydrogen also their physiologically compatible salts with bases.

4. Use according to claim 3, wherein the inhibitor of the progesterone receptor is the compound 11β-(4-acetylphenyl)-17β-hydroxy-17α-(1,1,2,2,2-pentafluoroethyl)estra-4,9-dien-3-one.

5. Use according to one of claims 1 to 4, wherein the tumor cells have a high and/or constitutive progesterone receptor expression.

6. Use according to one of claims 1 to 5, wherein the tumor cells are breast carcinoma cells.

7. Use according to one of claims 1 to 6, wherein the binding of EGF and/or other factors, which bind to the EGF receptor, is inhibited in tumor cells.

8. Use according to one of claims 1 to 7, wherein the formation of heterodimers between the EGF receptor and erbB2 is inhibited.

9. Use according to one of claims 1 to 8 for tumor therapy.

10. Use according to claim 9 to inhibit the progression of a tumor of steroid-dependent growth to growth-factor-dependent growth.
